(19) United States
(12) Patent Application Publication
(10) Pub. No.: US 2006/0194782 A1
(43) Pub. Date: Aug. 31, 2006

(54) Pharmacologically active imidazo[4,5-c] pyridines

(75) Inventor: M. Vittoria Chiesa, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/571,571

(22) PCT Filed: Sep. 17, 2004
(86) PCT No.: PCT/EP04/52229

§ 371 Date: Mar. 10, 2006

(30) Foreign Application Priority Data
Sep. 18, 2003 (EP) 03021087.6

(51) Int. Cl. A61K 31/5377; A61K 31/496; A61K 31/4745; C07D 471/02

(52) U.S. Cl. 514/210.21; 546/118; 544/125; 544/362; 514/234.2; 514/253.04; 514/303

(57) ABSTRACT

The invention relates to imidazo[4,5-c]pyridines of formula 1, 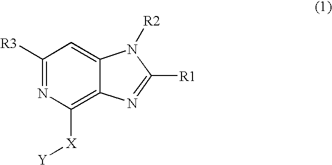 in which the substituents and symbols have the meanings indicated in the description. The compounds have gastric secretion inhibiting and excellent gastric and intestinal protective action properties.

### TECHNICAL FIELD

The invention relates in a first aspect to the use of compounds for the prevention and treatment of gastrointestinal disorders. The invention further relates to novel compounds, which are used in the pharmaceutical industry as active compounds for the production of medicaments.

### PRIOR ART

The European Patent Application EP 0082369 (=U.S. Pat. No. 4,654,350) describes imidazo[4,5-c]pyridines which are said to possess selective GABA-agonistic and/or benzodiazepine-antagonistic as well as anorectic properties.

The European Patent Application EP 0038568 describes a method for the preparation of 4-substituted-1β-D-ribofuranosyl-1H-imidazo-[4,5-c]pyridines by enzymatic ribosylation of the corresponding 4-substituted 1H-imidazo-[4,5-c]pyridines.

The International Patent Application WO 03/066586 describes 2,7-disubstituted purines and their isosteres such as the corresponding 3-deazapurines and 3-deaza-8-azapurines. As starting materials or intermediates for the synthesis of these compounds, 4-benzyloxy-6-chloroimidazo[4,5-c]pyridines are described.

In the European Patent Application EP 0033094, the U.S. Pat. No. 6,613,775 and the International Patent Application WO 98/37080 (=U.S. Pat. No. 6,265,415) imidazo[1,2-a]pyridine compounds are disclosed, which are said to be effective inter alia as inhibitors of the gastrointestinal H+/K+-ATPase and thereby as inhibitors of gastric acid secretion.

In the European Patent Application EP 0356234 (=U.S. Pat. No. 5,047,411) different benzazole compounds are disclosed, which are said to exhibit antiulcer activity and H2-receptor antagonism.

The International Patent Application WO 79/00206 (=U.S. Pat. No. 4,195,088) describes substituted derivatives of 1,3-dihydro-imidazo[4,5-b]pyridine-2-ones, which compounds are said to have gastric antisecretory, antiulcerous and anorexic activities.

In the international patent application WO91/17163, imidazo[4,5-c]pyridine derivatives having a very specific substitution pattern are disclosed, which are said to be suitable inter alia for the treatment of gastrointestinal distress.

### SUMMARY OF THE INVENTION

A first aspect of the invention (aspect 1) relates to the use of compounds of the formula 1, 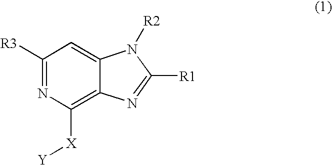
in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4Calkoxy-1-4C-alkoxy or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
   wherein
   Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   or Y denotes the group gp 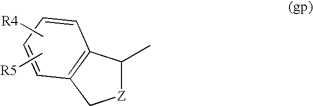
   wherein
   Z has the meaning —CHR8- or —CHR8-CHR9-
   where in Ar and/or in the group gp
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds
      for the prevention and treatment of gastrointestinal disorders.

A second aspect of the invention (aspect 2) relates to the use of compounds of the formula 1 as described for aspect 1 for the production of medicaments for the prevention and treatment of gastrointestinal disorders.

A third aspect of the invention (aspect 3) relates to compounds of the formula 1 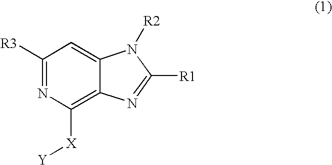
in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4Calkoxy-1-4C-alkoxy or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
   wherein
   Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   or Y denotes the group gp 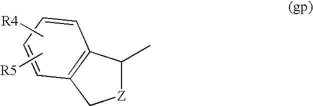
   wherein
   Z has the meaning —CHR8- or —CHR8-CHR9-
   where in Ar and/or in the group gp
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      with the proviso that R1 does not have the meaning hydrogen when
R2 is hydrogen, 1-4C-alkyl, 1-4C-alkoxy-1-4C-alkyl or cyclopropylmethyl,
R3 is hydrogen or halogen,
X is O (oxygen) or NH,
Y denotes CH₂—Ar
wherein
Ar is phenyl, naphtyl or benzothienyl substituted by R4 and R5
wherein
R4 is hydrogen, halogen, trifluoromethyl or nitro,
R5 is hydrogen, halogen or trifluoromethyl,
   and the salts of these compounds.

Halogen within the meaning of the invention is bromo, chloro and fluoro.

1-4C-Alkyl represents a straight-chain or branched alkyl group having 1 to 4 carbon atoms. Examples which may be mentioned are the butyl, isobutyl, sec-butyl, tert-butyl, propyl, isopropyl, ethyl and the methyl group.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkyl-1-4C-alkyl represents one of the aforementioned 1-4C-alkyl groups, which is substituted by one of the aforementioned 3-7C-cycloalkyl groups. Examples which may be mentioned are the cyclopropylmethyl, the cyclohexylmethyl and the cyclohexylethyl group.

1-4C-Alkoxy represents a group, which in addition to the oxygen atom contains one of the aforementioned 1-4C-alkyl groups. Examples which may be mentioned are the butoxy, isobutoxy, sec-butoxy, tert-butoxy, propoxy, isopropoxy and preferably the ethoxy and methoxy group.

1-4C-Alkoxy-1-4C-alkyl represents one of the aforementioned 1-4C-alkyl groups, which is substituted by one of the aforementioned 1-4C-alkoxy groups. Examples which may be mentioned are the methoxymethyl, the methoxyethyl group and the butoxyethyl group.

1-4C-Alkoxycarbonyl (1-4C-alkoxy-CO—) represents a carbonyl group, to which one of the aforementioned 1-4C-alkoxy groups is bonded. Examples which may be mentioned are the methoxycarbonyl (CH₃O—C(O)—) and the ethoxycarbonyl group (CH₃CH₂O—C(O)—).

2-4C-Alkenyl represents a straight-chain or branched alkenyl group having 2 to 4 carbon atoms. Examples which may be mentioned are the 2-butenyl, 3-butenyl, 1-propenyl and the 2-propenyl group (allyl group).

2-4C-Alkynyl represents a straight-chain or branched alkynyl group having 2 to 4 carbon atoms. Examples which may be mentioned are the 2-butynyl, 3-butynyl, and preferably the 2-propynyl, group (propargyl group).

Fluoro-1-4C-alkyl represents one of the aforementioned 1-4C-alkyl groups, which is substituted by one or more fluorine atoms. An example which may be mentioned is the trifluoromethyl group.

Hydroxy-1-4C-alkyl represents one of the aforementioned 1-4C-alkyl groups, which is substituted by a hydroxy group. Examples which may be mentioned are the hydroxymethyl, the 2-hydroxyethyl and the 3-hydroxypropyl group.

Mono- or di-1-4C-alkylamino represents an amino group, which is substituted by one or by two—identical or different—groups from the aforementioned 1-4C-alkyl groups. Examples which may be mentioned are the dimethylamino, the diethylamino and the diisopropylamino group.

Mono- or di-1-4C-alkylamino-1-4C-alkylcarbonyl represents a 1-4C-alkylcarbonyl group, which is substituted by a mono- or di-1-4C-alkylamino groups. Examples, which may be mentioned, are the dimethylamino-methylcarbonyl and the dimethylamino-ethylcarbonyl group.

Fluoro-2-4C-alkyl represents a 2-4C-alkyl group, which is substituted by one or more fluorine atoms. An example which may be mentioned is the 2,2,2-trifluoroethyl group.

1-4C-Alkoxy-1-4C-alkoxy represents one of the aforementioned 1-4C-alkoxy groups, which is substituted by a further 1-4C-alkoxy group. Examples which may be mentioned are the groups 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl represents one of the aforementioned 1-4C-alkoxy-1-4C-alkyl groups, which is substituted by one of the aforementioned 1-4C-alkoxy groups. An example which may be mentioned is the group 2-(methoxy)ethoxymethyl (CH₃—O—CH₂—CH₂—O—CH_).

Fluoro-1-4C-alkoxy represents one of the aforementioned 1-4C-alkoxy groups, which is completely or mainly substituted by fluorine, “mainly” meaning in this connection that more than half of the hydrogen atoms are replaced by fluorine atoms. Examples of completely or mainly fluoro-substituted 1-4C-alkoxy groups which may be mentioned are the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy group

Fluoro-1-4C-alkoxy-1-4C-alkyl represents one of the aforementioned 1-4C-alkyl groups, which is substituted by a fluoro-1-4C-alkoxy group. Examples of fluoro-1-4C-alkoxy-1-4C-alkyl groups are the 1,1,2,2-tetra-fluoroethoxymethyl, the 2,2,2-trifluoroethoxymethyl, the trifluoromethoxyethyl and the difluoromethoxyethyl group.

1-7C-Alkyl represents a straight-chain or branched alkyl group having 1 to 7 carbon atoms. Examples which may be mentioned are the heptyl, isoheptyl (5-methylhexyl), hexyl, isohexyl (4-methylpentyl), neohexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl and the methyl group.

Groups Ar which may be mentioned are, for example, the following substituents: 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis(trifluoromethyl)phenyl, 4-butoxyphenyl, 2-chlorophenyl, 3-chlorophenyl, 4-chlorophenyl, 2-chloro-6-fluorophenyl, 3-chloro-4-fluoro phenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorphenoxy)-4-pyrazolyl, 1-methyl-3-trifluomethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)₂-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidinyl, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.

2-4C-Alkenyloxy represents a group, which in addition to the oxygen atom contains one of the abovementioned 2-4C-alkenyl groups. Examples, which may be mentioned, are the 2-butenyloxy, 3-butenyloxy, 1-propenyloxy and the 2-propenyloxy group (allyloxy group).

1-4C-Alkylcarbonyl represents a group, which in addition to the carbonyl group contains one of the abovementioned 1-4C-alkyl groups. An example which may be mentioned is the acetyl group.

Carboxy-1-4C-alkyl represents a 1-4C-alkyl group which is substituted by a carboxyl group. Examples, which may be mentioned, are the carboxymethyl and the 2-carboxyethyl group.

1-4C-Alkoxycarbonyl-1-4C-alkyl represents a 1-4C-alkyl group, which is substituted by one of the abovementioned 1-4C-alkoxycarbonyl groups. Examples, which may be mentioned, are the Methoxycarbonylmethyl and the ethoxycarbonylmethyl group.

Aryl-1-4C-alkyl represents one of the aforementioned 1-4C-alkyl groups, which is substituted by one of the abovementioned aryl groups. An exemplary preferred aryl-1-4C-alkyl group is the benzyl group.

Aryl-1-4C-alkoxy represents one of the aforementioned 1-4C-alkoxy groups, which is substituted by one of the abovementioned aryl groups. An exemplary preferred aryl-1-4C-alkoxy group is the benzyloxy group.

1-4C-Alkylcarbonylamino represents an amino group to which a 1-4C-alkylcarbonyl group is bonded. Examples which may be mentioned are the propionylamino (C₃H₇C(O)NH—) and the acetylamino group (acetamido group) (CH₃C(O)NH—).

1-4C-Alkoxycarbonylamino represents an amino group, which is substituted by one of the aforementioned 1-4C-alkoxycarbonyl groups. Examples, which may be mentioned, are the ethoxycarbonylamino and the methoxycarbonylamino group.

1-4C-Alkoxy-1-4C-alkoxycarbonyl represents a carbonyl group, to which one of the aforementioned 1-4C-alkoxy-1-4C-alkoxy groups is bonded. Examples which may be mentioned are the 2-(methoxy)ethoxycarbonyl (CH₃—O—CH₂CH₂—O—CO—) and the 2-(ethoxy)ethoxycarbonyl group (CH₃CH₂—O—CH₂CH₂—O—CO—).

1-4C-Alkoxy-1-4C-alkoxycarbonylamino represents an amino group, which is substituted by one of the aforementioned 1-4C-alkoxy-1-4C-alkoxycarbonyl groups. Examples which may be mentioned are the 2-(methoxy)ethoxycarbonylamino and the 2-(ethoxy)ethoxycarbonylamino group.

2-7C-Alkenyl represents straight-chain or branched alkenyl groups having 2 to 7 carbon atoms. Examples which may be mentioned are the 2-butenyl, 3-butenyl, 1-propenyl, the 2-propenyl (allyl) and the vinyl group. The aforementioned 2-4C-alkenyl groups are preferred.

Oxo-substituted 1-4C-alkoxy represents a 1-4C-alkoxy group, which instead of a methylene group contains a carbonyl group. An example which may be mentioned is the 2-oxopropoxy group.

3-7C-Cycloalkoxy represents cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkyl-1-4C-alkoxy represents one of the aforementioned 1-4C-alkoxy groups, which is substituted by one of the aforementioned 3-7C-cycloalkyl groups. Examples which may be mentioned are the cyclopropylmethoxy, the cyclobutylmethoxy and the cyclohexylethoxy group.

Hydroxy-1-4C-alkoxy represents aforementioned 1-4C-alkoxy groups, which are substituted by a hydroxy group. A preferred example which may be mentioned is the 2-hydroxyethoxy group. 1-4C-Alkoxy-1-4C-alkoxy-1-4C-alkoxy represents one of the aforementioned 1-4C-alkoxy groups, which is substituted by one of the aforementioned 1-4C-alkoxy-1-4C-alkoxy groups. A preferred example which may be mentioned is the methoxyethoxyethoxy group.

3-7C-Cycloalkoxy-1-4C-alkoxy represents one of the aforementioned 1-4C-alkoxy groups, which is substituted by one of the aforementioned 3-7C-cycloalkoxy groups. Examples which may be mentioned are the cyclopropoxymethoxy, the cyclobutoxymethoxy and the cyclohexyloxyethoxy group.

3-7C-Cycloalkyl-1-4C-alkoxy-1-4C-alkoxy represents one of the aforementioned 1-4C-alkoxy groups, which is substituted by one of the aforementioned 3-7C-cycloalkyl-1-4C-alkoxy groups. Examples which may be mentioned are the cyclopropylmethoxyethoxy, the cyclobutylmethoxyethoxy and the cyclohexylethoxyethoxy group.

1-4C-Alkylcarbonyloxy represents a 1-4C-alkylcarbonyl group which is bonded to an oxygen atom. An example which may be mentioned is the acetoxy group (CH₃CO—O—).

Halo-1-4C-alkoxy represents 1-4C-alkoxy groups which are completely or mainly substituted by halogen. “Mainly” in this connection means that more than half of the hydrogen atoms in the 1-4C-alkoxy groups are replaced by halogen atoms. Halo-1-4C-alkoxy groups are primarily chloro- and/or in particular fluoro-substituted 1-4C-alkoxy groups. Examples of halogen-substituted 1-4C-alkoxy groups which may be mentioned are the 2,2,2-trichloroethoxy, the hexachloroisopropoxy, the pentachloroisopropoxy, the 1,1,1-trichloro-3,3,3-trifluoro-2-propoxy, the 1,1,1-trichloro-2-methyl-2-propoxy, the 1,1,1-trichloro-2-propoxy, the 3-bromo-1,1,1-trifluoro-2-propoxy, the 3-bromo-1,1-trifluoro-2-butoxy, the 4-bromo-3,3,4,4-tetrafluoro-1-butoxy, the chlorodifluoromethoxy, the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy group.

Mono- or di-1-4C-alkylamino-1-4C-alkylcarbonyloxy represents a 1-4C-alkylcarbonyloxy group, which is substituted by one of the aforementioned mono- or di-1-4C-alkylamino groups. Examples, which may be mentioned, are the dimethylamino-methylcarbonyloxy and the dimethylamino-ethylcarbonyloxy group.

Possible salts of compounds of the formula 1—depending on substitution—are especially all acid addition salts. Particular mention may be made of the pharmacologically tolerable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are used in salt preparation—depending on whether a mono- or polybasic acid is concerned and on which salt is desired—in an equimolar quantitative ratio or one differing therefrom.

Pharmacologically intolerable salts, which can initially be obtained, for example, as process products in the production of the compounds according to the invention on the industrial scale, are converted into the pharmacologically tolerable salts by processes known to the person skilled in the art.

It is known to the person skilled in the art that the compounds according to invention and their salts, if, for example, they are isolated in crystalline form, can contain various amounts of solvents. The invention therefore also comprises all solvates and in particular all hydrates of the compounds of the formula 1, and also all solvates and in particular all hydrates of the salts of the compounds of the formula 1.

One embodiment (embodiment A) of aspect 1 of the invention relates to the use of compounds of the formula 1,

in which

R1 is hydrogen,
R2 is hydrogen, 1-4C-alkyl, 1-4C-alkoxy-1-4C-alkyl or cyclopropylmethyl,
R3 is hydrogen or halogen,
X is O (oxygen) or NH,
Y denotes CH₂—Ar
wherein
Ar is phenyl, naphtyl or benzothienyl substituted by R4 and R5
wherein
R4 is hydrogen, halogen, trifluoromethyl or nitro,
R5 is hydrogen, halogen or trifluoromethyl,
   and the salts of these compounds
   for the prevention and treatment of gastrointestinal disorders.

Another embodiment (embodiment B) of aspect 1 of the invention relates to the use of compounds of the formula 1 according to aspect 3 of the invention for the prevention and treatment of gastrointestinal disorders.

One embodiment (embodiment I) of aspect 2 of the invention relates to the use of compounds of the formula 1 according to embodiment A for the production of medicaments for the prevention and treatment of gastrointestinal disorders.

Another embodiment (embodiment II) of aspect 2 of the invention relates to the use of compounds of the formula 1 according to aspect 3 of the invention for the production of medicaments for the prevention and treatment of gastrointestinal disorders.

One embodiment (embodiment a) of aspect 3 of the invention relates to compounds of the formula 1, in which R1 is hydrogen and R2, R3, X and Y have the meanings as indicated in the outset, and the salts of these compounds.

Another embodiment (embodiment b) of aspect 3 of the invention relates to compounds of the formula 1, in which R1 is 1-4C-alkyl and R2, R3, X and Y have the meanings as indicated in the outset, and the salts of these compounds.

Another embodiment (embodiment c) of aspect 3 of the invention relates to compounds of the formula 1, in which R1 is halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino, and R2, R3, X and Y have the meanings as indicated in the outset, and the salts of these compounds.

Another embodiment (embodiment d) of aspect 3 of the invention relates to compounds of the formula 1, in which X is O (oxygen) and R1, R2, R3 and Y have the meanings as indicated in the outset, and the salts of these compounds.

Another embodiment (embodiment e) of aspect 3 of the invention relates to compounds of the formula 1, in which X is NH and R1, R2, R3 and Y have the meanings as indicated in the outset, and the salts of these compounds.

The invention also relates to compounds of the formula 1,

in which

R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
   wherein
   Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   or Y denotes the group gp 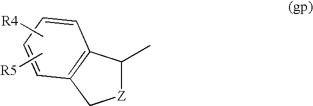
   wherein
   Z has the meaning —CHR8- or —CHR8-CHR9-
   where in Ar and/or in the group gp
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      with the proviso that R1 does not have the meaning hydrogen when
R2 is hydrogen, 1-4C-alkyl, 1-4C-alkoxy-1-4C-alkyl or cyclopropylmethyl,
R3 is hydrogen or halogen,
X is O (oxygen) or NH,
Y denotes CH₂—Ar
wherein
Ar is phenyl, naphtyl or benzothienyl substituted by R4 and R5
wherein
R4 is hydrogen, halogen, trifluoromethyl or nitro,
R5 is hydrogen, halogen or trifluoromethyl,
   and the salts of these compounds.

The invention also relates to compounds of the formula 1

in which

R1 is halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4C-alkoxy-1-4C-alkoxy or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
   wherein
   Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   or Y denotes the group gp 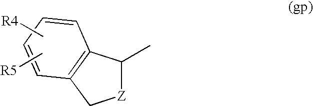
   wherein
   Z has the meaning —CHR8- or —CHR8-CHR9-
   where in Ar and/or in the group gp
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

Preferred compounds of the formula 1 are those compounds, in which
R1 is hydrogen or 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy-1-4C-alkoxy-1-4C-alkyl, 1-4C-alkoxy, 1-4C-alkoxy-1-4C-alkoxy or the group —CO—NR31R32,
   where
   R31 is hydrogen, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
   wherein
   Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   or Y denotes the group gp 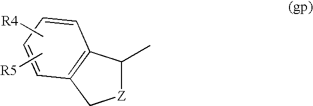
   wherein
   Z has the meaning —CHR8- or —CHR8-CHR9-
   where in Ar and/or in the group gp
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      with the proviso that R1 does not have the meaning hydrogen when
R2 is hydrogen or 1-4C-alkyl,
R3 is hydrogen or halogen
X is O (oxygen) or NH,
Y denotes CH₂—Ar
wherein
Ar is phenyl, naphtyl or benzothienyl substituted by R4 and R5
wherein
R4 is hydrogen, halogen, trifluoromethyl or nitro,
R5 is hydrogen, halogen or trifluoromethyl,
   and the salts of these compounds.

Still preferred compounds of the formula 1 are those compounds, in which
R1 is 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy-1-4C-alkoxy-1-4C-alkyl, 1-4C-alkoxy, 1-4C-alkoxy-1-4C-alkoxy or the group —CO—NR31R32,
   where
   R31 is hydrogen, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperdino, piperazino, N-1-4C-alkylpiperazino, morpholino, aziridino or azetidino group,
X is O (oxygen) or NH and
Y has either the meaning —CH₂—Ar
   wherein
   Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   or Y denotes the group gp 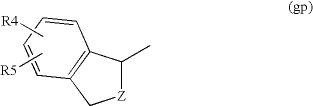
   wherein
   Z has the meaning —CHR8- or —CHR8-CHR9-
   where in Ar and/or in the group gp
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylaniino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

Still preferred compounds of the formula 1 are those compounds, in which
R1 is hydrogen or 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy-1-4C-alkoxy-1-4C-alkyl, 1-4C-alkoxy, 1-4C-alkoxy-1-4C-alkoxy or the group —CO—NR31R32,
   where
   R31 is hydrogen, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
   wherein
   Ar is a phenyl substituted by R4 and R5
      or Y denotes the group gp 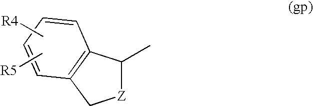
   wherein
   Z has the meaning —CHR8-
   where in Ar and/or in the group gp
   R4 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
   R5 is hydrogen, 1-4C-alkyl, 1-4C-alkoxy or halogen,
   R8 is hydrogen, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
      with the proviso that R1 does not have the meaning hydrogen when
R2 is hydrogen or 1-4C-alkyl,
R3 is hydrogen or halogen,
X is O (oxygen) or NH,
Y denotes CH₂—Ar
wherein
Ar is phenyl substituted by R4 and R5
wherein
R4 is hydrogen, halogen or trifluoromethyl,
R5 is hydrogen or halogen,
   and the salts of these compounds.

Still preferred compounds of the formula 1 are those compounds, in which
R1 is 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy-1-4C-alkoxy-1-4C-alky, 1-4C-alkoxy, 1-4C-alkoxy-1-4C-alkoxy or the group —CO—NR31R32,
   where
   R31 is hydrogen, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
   wherein
   Ar is a phenyl substituted by R4 and R5
      or Y denotes the group gp 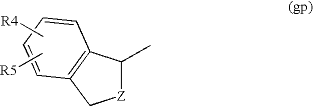
   wherein
   Z has the meaning —CHR8-
   where in Ar and/or in the group gp
   R4 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
   R5 is hydrogen, 1-4C-alkyl, 1-4C-alkoxy or halogen,
   R8 is hydrogen, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
      and the salts of these compounds.

Still preferred compounds of the formula 1 are those compounds, in which
R1 is 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy, 1-4C-alkoxy-1-4C-alkoxy or the group —CO—NR31R32,
   where
   R31 is hydrogen, 1-4C-alkyl, hydroxy-1-4C-alkyl or 1-4C-alkoxy-1-4C-alkyl and
   R32 is hydrogen or 1-4C-alkyl,
X is O (oxygen) or NH and
Y has either the meaning —CH₂—Ar
   wherein
   Ar is a phenyl substituted by R4 and R5
      or Y denotes the group gp 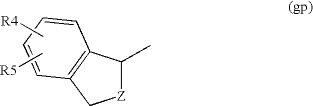
   wherein
   Z has the meaning —CHR8-
   where in Ar and/or in the group gp
   R4 is hydrogen, 1-4C-alkyl, 1-4C-alkoxy, halogen or 1-4C-alkylcarbonylamino
   R5 is hydrogen, 1-4C-alkyl or halogen,
   R8 is hydrogen or hydroxyl,
      and the salts of these compounds.

Compounds of the formula 1 which are to be emphasized are those compounds,

in which

R1 is 1-4C-alkyl,
R2 is hydrogen or 1-4C-alkyl,
R3 is hydrogen, halogen, carboxyl, 1-4C-alkoxycarbonyl, 1-4C-alkoxy, 1-4C-alkoxy-1-4C-alkoxy or the group —CO—NR31R32,
   where
   R31 is hydrogen, 1-4C-alkyl or hydroxy-1-4C-alkyl and
   R32 is hydrogen or 1-4C-alkyl,
X is O (oxygen) or NH and
Y has either the meaning —CH₂—Ar
   wherein
   Ar is a phenyl substituted by R4 and R5,
      or Y denotes the group gp 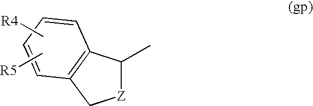
   wherein
   Z has the meaning —CHR8-,
   where in Ar and/or in the group gp
   R4 is hydrogen, 1-4C-alkyl or halogen,
   R5 is hydrogen or 1-4C-alkyl,
   R8 is hydrogen
      and the salts of these compounds.

In one aspect, the invention relates to compounds of the formula 1a 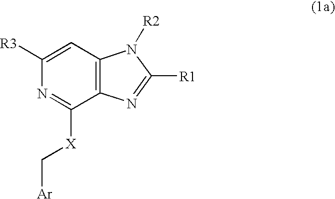
in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4C-alkoxy-1-4C-alkoxy or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   where
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      with the proviso that R1 does not have the meaning hydrogen when
R2 is hydrogen, 1-4C-alkyl, 1-4C-alkoxy-1-4C-alkyl or cyclopropylmethyl,
R3 denotes hydrogen or halogen,
X is O (oxygen) or NH,
Ar is phenyl, naphtyl or benzothienyl substituted by R4 and R5
wherein
R4 is hydrogen, halogen, trifluoromethyl or nitro,
R5 is hydrogen, halogen or trifluoromethyl,
   and the salts of these compounds.

The invention further relates to compounds of the formula 1a,

in which

R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   where
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      with the proviso that R1 does not have the meaning hydrogen when
R3 denotes hydrogen or halogen and
Ar is phenyl, naphtyl or benzothienyl substituted by R4 and R5
wherein
R4 is hydrogen, halogen, trifluoromethyl or nitro,
R5 is hydrogen, halogen or trifluoromethyl,
   and the salts of these compounds.

The invention further relates to compounds of the formula 1a

in which

R1 is halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4C-alkoxy-1-4C-alkoxy or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   where
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

Another aspect of the invention relates to compounds of the formula 1b 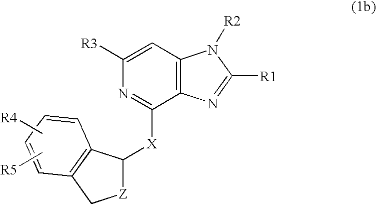
in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4C-alkoxy-1-4C-alkoxy or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
X is O (oxygen) or NH and
Z has the meaning —CHR8- or —CHR8-CHR9-
   where
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycfoalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

The invention further relates to compounds of the formula 1b,

in which

R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
X is O (oxygen) or NH and
Z has the meaning —CHR8- or —CHR8-CHR9-
   where
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

The compounds of the formula 1b have up to three chiral centers in the parent structure. The invention thus relates to all conceivable stereoisomers in any desired mixing ratio to one another, including the pure enantiomers, which are a preferred subject of the invention.

Among the compounds of the formula 1a, preferred compounds are those of the formula 1a-1 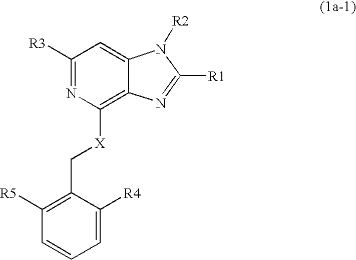
in which
R1 is hydrogen or 1-4C-alkyl,
R2 is hydrogen or 1-4C-alkyl,
R3 is hydrogen, halogen, carboxyl, 1-4C-alkoxycarbonyl, hydroxy-1-4C-alkyl, 1-4C-alkoxy-1-4C-alkyl, 1-4C-alkoxy, 1-4C-alkoxy-1-4C-alkoxy or the group —CO—NR31R32,
   where
   R31 is hydrogen, 1-7C-alkyl, hydroxy-1-4C-alkyl or 1-4C-alkoxy-1-4C-alkyl and
   R32 is hydrogen or 1-7C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl, 1-4C-alkoxy or halogen and
X is O (oxygen) or NH,
   with the proviso that R1 does not have the meaning hydrogen when
R2 is hydrogen or 1-4C-alkyl,
R3 is hydrogen or halogen,
X is O (oxygen) or NH,
R4 is hydrogen, halogen or trifluoromethyl,
R5 is hydrogen or halogen,
   and the salts of these compounds.

Further preferred compounds of the formula 1a are those compounds of the formula 1a-1,

in which

R1 is hydrogen or 1-4C-alkyl,
R2 is hydrogen or 1-4C-alkyl,
R3 is hydrogen, carboxyl, 1-4C-alkoxycarbonyl, hydroxy-1-4C-alkyl, 1-4C-alkoxy-1-4C-alkyl, or the group —CO—NR31R32,
   where
   R31 is hydrogen, 1-7C-alkyl, hydroxy-1-4C-alkyl or 1-4C-alkoxy-1-4C-alkyl and
   R32 is hydrogen or 1-7C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl, 1-4C-alkoxy or halogen and
X is O (oxygen) or NH,
   with the proviso that R1 does not have the meaning hydrogen when
R3 denotes hydrogen,
R4 is hydrogen, halogen or trifluoromethyl,
R5 is hydrogen or halogen,
   and the salts of these compounds.

Still further preferred compounds of the formula 1a are those compounds of the formula 1a-1,

in which

R1 is 1-4C-alkyl,
R2 is hydrogen or 1-4C-alkyl,
R3 is hydrogen, halogen, carboxyl, 1-4C-alkoxycarbonyl, hydroxy-1-4C-alkyl, 1-4C-alkoxy-1-4C-alkyl, 1-4C-alkoxy, 1-4C-alkoxy-1-4C-alkoxy or the group —CO—NR31R32,
   where
   R31 is hydrogen, 1-7C-alkyl, hydroxy-1-4C-alkyl or 1-4C-alkoxy-1-4C-alkyl and
   R32 is hydrogen or 1-7C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl, 1-4C-alkoxy or halogen and
X is O (oxygen) or NH,
   and the salts of these compounds.

Preferred compounds of the formula 1a-1 are those, in which
R1 is 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy, 1-4C-alkoxy-1-4C-alkoxy or the group —CO—NR31R32,
   where
   R31 is hydrogen, 1-4C-alkyl, hydroxy-1-4C-alkyl or 1-4C-alkoxy-1-4C-alkyl and
   R32 is hydrogen or 1-4C-alkyl,
R4 is hydrogen, 1-4C-alkyl, halogen or 1-4C-alkylcarbonylamino,
R5 is hydrogen, 1-4C-alkyl or halogen,
X is O (oxygen) or NH,
   and their salts.

Still preferred compounds of the formula 1a-1 are those, in which
R1 is 1-4C-alkyl,
R2 is 1-4C-alkyl,
R3 is hydrogen, carboxyl, 1-4C-alkoxycarbonyl, 1-4C-alkoxy-1-4C-alkyl or the group —CO—NR31R32,
   where
   R31 is hydrogen, 1-4C-alkyl, hydroxy-1-4C-alkyl or 1-4C-alkoxy-1-4C-alkyl and
   R32 is hydrogen or 1-4C-alkyl,
R4 is 1-4C-alkyl, halogen or 1-4C-alkylcarbonylamino,
R5 is 1-4C-alkyl or halogen,
X is O (oxygen) or NH,
   and their salts.

Compounds of the formula 1a-1 which are to be emphasized are those compounds, in which
R1 is 1-4C-alkyl,
R2 is hydrogen or 1-4C-alkyl,
R3 is hydrogen, halogen, carboxyl, 1-4C-alkoxycarbonyl, 1-4C-alkoxy, 1-4C-alkoxy-1-4C-alkoxy or the group —CO—NR31R32,
   where
   R31 is hydrogen, 1-4C-alkyl or hydroxy-1-4C-alkyl and
   R32 is hydrogen or 1-4C-alkyl,
R4 is hydrogen, 1-4C-alkyl or halogen
R5 is hydrogen, 1-4C-alkyl or halogen,
X is O (oxygen) or NH,
   and their salts.

Compounds of the formula 1a-1 which are also to be emphasized are those compounds, in which
R1 is 1-4C-alkyl,
R2 is 1-4C-alkyl,
R3 is hydrogen or the group —CO—NR31R32,
   where
   R31 is hydrogen, 1-4C-alkyl or hydroxy-1-4C-alkyl and
   R32 is hydrogen or 1-4C-alkyl,
R4 is 1-4C-alkyl
R5 is 1-4C-alkyl
X is NH,
   and their salts.

Among the compounds of the formula 1b, compounds of the formula 1 b-1 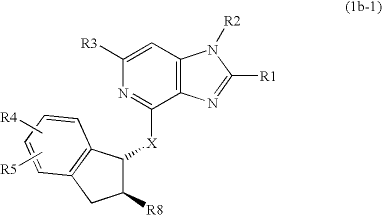
are preferred.

Preferred compounds of the formula 1 b-1 are those, in which
R1 is hydrogen or 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy-1-4C-alkoxy-1-4C-alkyl, 1-4C-alkoxy, 1-4C-alkoxy-1-4C-alkoxy or the group —CO—NR31R32,
   where
   R31 is hydrogen, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen,
R8 is hydrogen, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
X is O (oxygen) or NH,
   and their salts.

Still preferred compounds of the formula 1b-1 are those, in which
R1 is 1-4C-alkyl,
R2 is hydrogen or 1-4C-alkyl,
R3 is carboxyl, 1-4C-alkoxycarbonyl, 1-4C-alkoxy-1-4C-alkyl, 1-4C-alkoxy-1-4C-alkoxy-1-4C-alkyl or the group —CO—NR31R32,
   where
   R31 is hydrogen, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen,
R8 is hydrogen, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
X is O (oxygen) or NH,
   and their salts.

Still preferred exemplary compounds of the formula 1b-1 are those, in which
R1 is 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy-1-4C-alkoxy-1-4C-alkyl, 1-4C-alkoxy, 1-4C-alkoxy-1-4C-alkoxy orthe group —CO—NR31R32,
   where
   R31 is hydrogen, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen,
R8 is hydrogen, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy or 1-4C-alkoxy-1-4C-alkoxy-1-4C-alkoxy
X is O (oxygen) or NH,
   and their salts

Still preferred exemplary compounds of the formula 1 b-1 are those, in which
R1 is 1-4C-alkyl,
R2 is hydrogen or 1-4C-alkyl,
R3 is carboxyl, 1-4C-alkoxycarbonyl, 1-4C-alkoxy-1-4C-alkyl, 1-4C-alkoxy-1-4C-alkoxy-1-4C-alkyl or the group —CO—NR31R32,
   where
   R31 is hydrogen, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen,
R8 is hydrogen, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy or 1-4C-alkoxy-1-4C-alkoxy-1-4C-alkoxy
X is O (oxygen) or NH,
   and their salts

Compounds of the formula 1 b-1 which are to be emphasized are those compounds, in which
R1 is 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy, 1-4C-alkoxy-1-4C-alkoxy or the group —CO—NR31R32,
   where
   R31 is hydrogen, 1-4C-alkyl, hydroxy-1-4C-alkyl or 1-4C-alkoxy-1-4C-alkyl and
   R32 is hydrogen or 1-4C-alkyl,
R4 is hydrogen, 1-4C-alkyl, 1-4C-alkoxy or halogen,
R5 is hydrogen,
R8 is hydrogen or hydroxyl,
X is O (oxygen) or NH,
   and their salts.

Compounds of the formula 1 b-1 which are to be particularly emphasized are those compounds, in which
R1 is 1-4C-alkyl,
R2 is 1-4C-alkyl,
R3 is hydrogen,
R4 is hydrogen,
R5 is hydrogen,
R8 is hydrogen,
X is O (oxygen),
   and their salts.

Preferred compounds are those of the formula 1a-1.

Particularly preferred are the compounds given as final products of formula 1 in the examples, and the salts of these compounds.

The compounds according to the invention can be synthesized from corresponding starting compounds, for example according to the reaction schemes given below. The synthesis is carried out in a manner known to the expert, for example as described in more detail in the following examples.

The starting compounds are known, for example from M. Nettekoven, C. Jenny, Org. Process Res. Dev. (2003), 7, 38-43 and U. Neumann, F. Vögtle, Chem. Ber. 1989, 122, 589-591 (2,6-Dibromo-4-nitro-pyridine). Compounds of the formula 3 (scheme 1) and 4 (scheme 2) can be prepared using analogous process steps known in literature (R. J. Rousseau, R. K. Robins, J. Het. Chem. (1965), 2, 196-201).

Compounds of the general formula 1a are obtained starting from substituted bromopyridines of the formula 2 (scheme 1 and scheme 2).

In general, if compounds of the formula 1a are desired where R3=1-4C-alkoxycarbonyl or R3=—CO—NR31R32, an appropriate derivatization can be performed in a manner known per se (e.g. metal catalysed carbonylation of the corresponding bromo derivate of the formula 3 at the last step of the synthesis as shown in scheme 1).

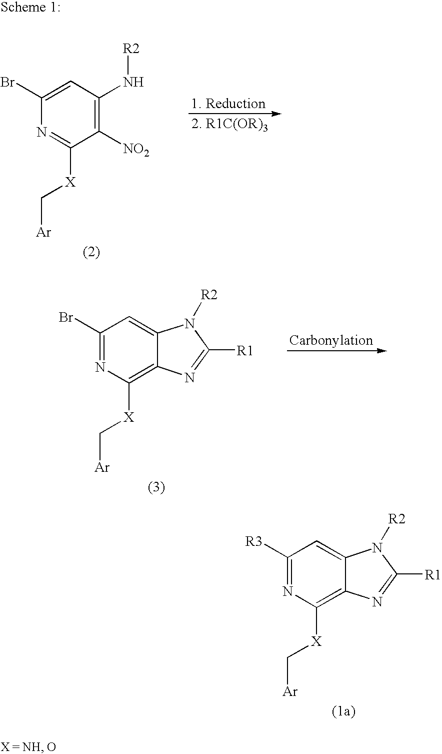

If compounds of the formula 1a are desired where R3=hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy or 1-4C-alkoxy-1-4C-alkoxy an appropriate derivatization can be performed in a manner known per se (e.g. nucleophilic substitution of the bromo substituent at the stage of the pyridine of formula 2) (scheme 2).

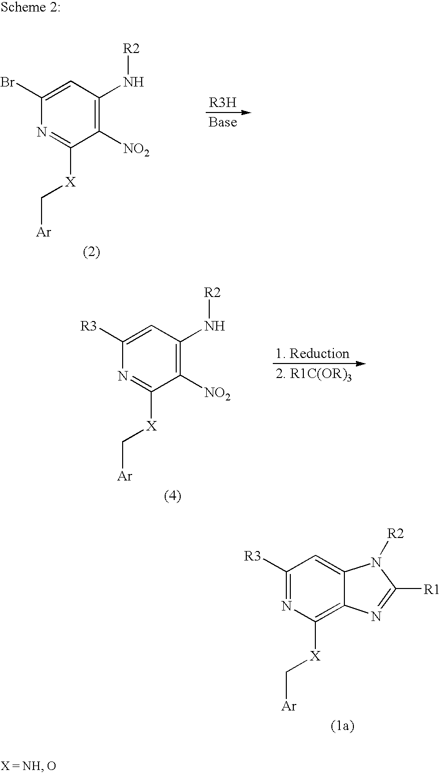

Analogously, compounds of the general formula 1b can be obtained by reacting substituted dibromopyridines of the formula 5 with bicyclic derivatives of the formula 6, carring any desired substituents R4, R5 and Z defined as described above, and further transformations as shown in scheme 3.

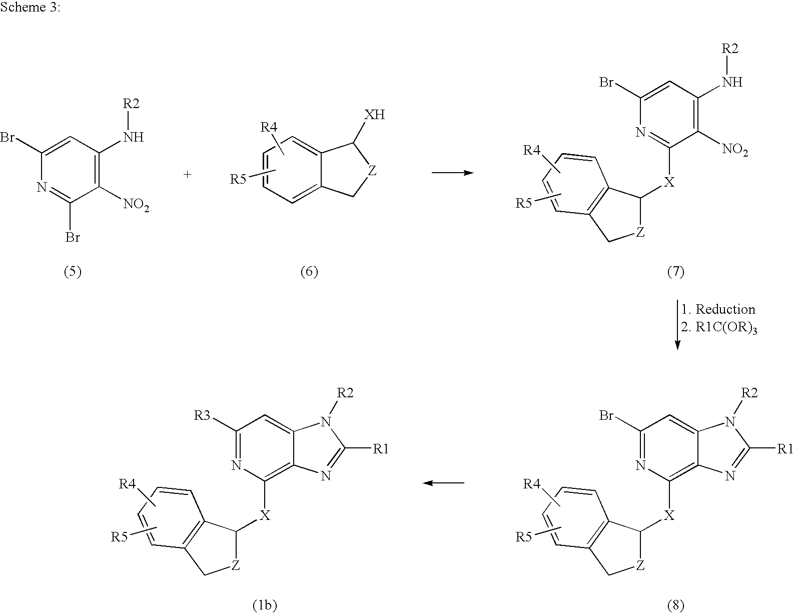

Alternatively, compounds of the formula 1 can be obtained starting from 4-chloro-1H-benzoimidazole derivatives of the formula 9 with suitable substituents R1, R2 and R3 as show n in scheme 4. Compounds of the formula 9 are known (see for example Rousseau, R. J.; Robins, R. K., J. Het. Chem. (1965), 2(2), 196-201 or Houston, D. M.; Dolence, E. K.; Keller, B. T.; Patel-Thombre, U.; Borchardt, R. T., J. Medicinal Chemistry (1985), 28(4), 467-71) or can be prepared in manner similar to that described in the examples.

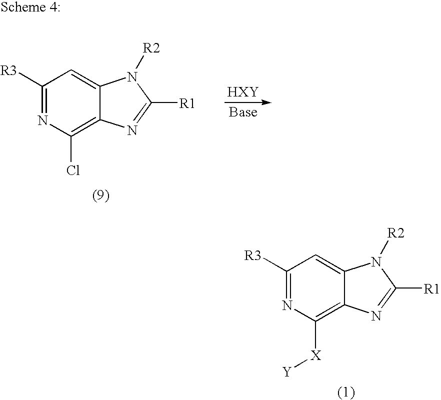

The reaction steps outlined above are carried out in a manner known per se, e.g. as described in more detail in the examples.

The following examples serve to illustrate the invention in greater detail without restricting it. Likewise, further compounds of the formula 1 whose preparation is not described explicitly can be prepared in an analogous manner or in a manner familiar per se to the person skilled in the art using customary process techniques. The abbreviation min stands for minute(s), h for hour(s) and m.p. for melting point.

### EXAMPLES

Final Products of Formula 1

### 1. (1,2-Dimethyl-1H-imidazo[4,5-c]pyridin-4-ylmethyl)-(2-ethyl-6-methyl-phenyl)-amine

A solution of 200 mg (0.53 mmol) (6-Bromo-1,2-dimethyl-1H-imidazo[4,5-c]pyridin-4-yl)-(2-ethyl-6-methyl-benzyl)-amine and 36.5 mg (0.91 mmol) sodium hydroxide in ethanol (20 ml) was hydrogenated over 40 mg 10% Pd/C (1 bar H₂) for 6 h. The catalyst was filtered off and the filtrate concentrated in vacuo. The residue was purified by column chromatography on silica gel (ethyl acetate) to afford 283.6 mg (79%) of the title compound as a beige solid. m.p. 141°-142° C.

### 2. 4-(2-Ethyl-6-methyl-benzylamino)-1,2-dimethyl-1H-imidazo[4,5-c]pyridine-6-carboxylic Acid Dimethylamide

To a solution of 295 mg (0.87 mmol) 4-(2-ethyl-6-methyl-benzylamino)-1,2-dimethyl-1H-imidazo[4,5-c]pyridine-6-carboxylic acid in 12 ml dichloromethane were added 430 mg (1.3 mmol) O-(1H-benzotriazol-1-yl)-N,N, N′,N′-tetramethyluronium tetrafluoroborate (TBTU) and the mixture was stirred at room temperature overnight. 1.8 ml (3.5 mmol) Dimethylamine (2M in THF) were added and the reaction mixture was stirred for 24 h at room temperature. The brown solution was poured into 20 ml water and extracted with dichloromethane (2×10 ml). The collected organic layers were washed with 2M NaOH solution (2×5 ml), the organic layers dried over magnesium sulphate and concentrated in vacuo. The residue was crystallized from diisopropyl ether to afford 236 mg (74%) of the title compound as a beige solid. m.p 204°-206° C.

### 3. 4-(2-Ethyl-6-methyl-benzylamino)-1,2-dimethyl-1H-imidazo[4,5-c]pyridine-6-carboxylic Acid Amide

To a solution of 295 mg (0.87 mmol) 4-(2-ethyl-6-methyl-benzylamino)-1,2-dimethyl-1H-imidazo[4,5-c]pyridine-6-carboxylic acid in 12 ml dichloromethane were added 430 mg (1.3 mmol) O-(1H-benzotriazol-1-yl)-N,N, N′,N′-tetramethyluronium tetrafluoroborate (TBTU) and the mixture was stirred at room temperature overnight. 10.4 ml (5.2 mmol) Ammonia (0.5M in dioxane) were added and the reaction mixture was stirred for 24 h at room temperature. The suspension was poured into 20 ml water and extracted with dichloromethane (2×10 ml). The collected organic layers were washed with 2M NaOH solution (2×5 ml), the organic layers dried over magnesium sulphate and concentrated in vacuo. The residue was crystallized from diisopropyl ether to afford 229 mg (72%) of the title compound as a beige solid. m.p 264°-266° C.

### 4. 4-(2-Ethyl-6-methyl-benzylamino)-1,2-dimethyl-1H-imidazo[4,5-c]pyridine-6-carboxylic Acid Methylamide

To a solution of 295 mg (0.87 mmol) 4-(2-ethyl-6-methyl-benzylamino)-1,2-dimethyl-1H-imidazo[4,5-c]pyridine-6-carboxylic acid in 12 ml dichloromethane were added 430 mg (1.3 mmol) O-(1H-benzotriazol-1-yl)-N,N, N′,N′-tetramethyluronium tetrafluoroborate (TBTU) and the mixture was stirred at room temperature overnight. 1.8 ml (3.5 mmol) Methylamine (2M in THF) were added and the reaction mixture was stirred for 24 h at room temperature. The brown solution was poured into 20 ml water and extracted with dichloromethane (2×10 ml). The collected organic layers were washed with 2M NaOH solution (2×5 ml), the organic layers dried over magnesium sulphate and concentrated in vacuo. The residue was crystallized from diisopropyl ether to afford 82 mg (26%) of the title compound as a beige solid. m.p 157°-160° C.

### 5. 4-(2-Ethyl-6-methyl-benzylamino)-1,2-dimethyl-1H-imidazo[4,5-c]pyridine-6-carboxylic Acid (2-Hydroxy-ethyl)-amide

To a solution of 295 mg (0.87 mmol) 4-(2-ethyl-6-methyl-benzylamino)-1,2-dimethyl-1H-imidazo[4,5-c]pyridine-6-carboxylic acid in 12 ml dichloromethane were added 430 mg (1.3 mmol) O-(1H-benzotriazol-1-yl)-N,N, N′,N′-tetramethyluronium tetrafluoroborate (TBTU) and the mixture was stirred at room temperature overnight. 263 μl (4.4 mmol) Ethanolamine were added and the reaction mixture was stirred for 24 h at room temperature. The suspension was poured into 20 ml water and extracted with dichloromethane (2×10 ml). The collected organic layers were washed with 2M NaOH solution (2×5 ml), the organic layers dried over magnesium sulphate and concentrated in vacuo. The residue was crystallized from diisopropyl ether to afford 242 mg (76%) of the title compound as a beige solid. m.p 208°-209° C.

### 6. 4Benzylamino-1,2-dimethyl-1H-imidazo[4,5c]pyridine

A solution of 0.32 g (1.7 mmol) 4-chloro-1,2-dimethyl-1H-imidazo[4,5-c]pyridine and 0.73 g (5.3 mmol) potassium carbonate in benzyl amine (8 ml) was heated at 180° C. overnight. The benzyl amine was removed in vacuo, the residue diluted with dichloromethane (50 ml) and poured into water (50 ml). The layers were separated, the water-layer was adjusted to pH=10 and extracted with dichloromethane (5×20 ml). The organic layers were dried over magnesium sulphate and concentrated in vacuo. The crystallization of the residue from diethyl ether yielded 0.27 g (61%) of title compound as a white solid. m.p. 125°-127° C.

### 7. 4-Benzyloxy-1,2-dimethyl-1H-imidazo[4,5-c]pyridine

To a solution of 0.5 g (2.7 mmol) 4-chloro-1,2-dimethyl-1H-imidazo[4,5-c]pyridine in tetrahydrofurane (5 ml) were added 0.43 ml (4.1 mmol) benzyl alcohol and 0.48 g (4.13 mmol) potassium tert-butoxide. The mixture was cooled to 20° C. and a solution of 0.07 g (0.2 mmol) 18-crown-6 in tetrahydrofurane (2 ml) was added dropwise. The reaction was heated to reflux and stirred for 5 h. The reaction mixture was cooled to room temperature, poured into a mixture of water (15 ml)-dichloromethane (50 ml) and neutralized with potassium dihydrogen phosphate. The layers were separated, the organic layer was dried over magnesium sulphate and concentrated in vacuo. The residue was purified on silica gel (ethyl acetate) to afford 0.49 g (70%) of the title compound as a yellow solid. m.p. 115°-116° C.

### 8. 4-(2-Chloro-benzyloxy)-1,2-dimethyl-1H-imidazo[4,5-c]pyridine

To a solution of 0.5 g (2.7 mmol) 4-chloro-1,2-dimethyl-1H-imidazo[4,5-c]pyridine and 72.6 mg (0.2 mmol) 18-crown-6 in 3 ml dimethyl sulfoxide was added dropwise a solution of 1.4 g (9.6 mmol) 2-chloro-benzyl alcohol and 1.1 g (9.6 mmol) potassium tert-butoxide in 7 ml dimethyl sulfoxide. The mixture was heated to 70° C. and stirred for 30 min. The reaction mixture was cooled to room temperature, poured into a mixture of water (25 ml)-dichloromethane (50 ml) and neutralized with potassium dihydrogen phosphate. The layers were separated, the organic layer was dried over magnesium sulphate and concentrated in vacuo. The residue was crystallized from cyclohexane to afford 0.48 g (61%) of the title compound as a white solid. m.p. 180°-181° C.

### 9. 4-(2-Methyl-benzyloxy)-1,2-dimethyl-1H-imidazo[4,5c]pyridine

To a solution of 0.5 g (2.7 mmol) 4-chloro-1,2-dimethyl-1H-imidazo[4,5-]pyridine and 72.6 mg (0.2 mmol) 18-crown-6 in 2 ml dimethyl sulfoxide was added dropwise a solution of 0.7 g (5.5 mmol) 2-methyl-benzyl alcohol and 0.6 g (5.5 mmol) potassium tert-butoxide in 5 ml dimethyl sulfoxide. The mixture was heated to 70° C. and stirred for 90 min. The reaction mixture was cooled to room temperature, poured into a mixture of water (30 ml)-dichloromethane (50 ml) and neutralized with potassium dihydrogen phosphate. The layers were separated, the water layer was extracted with dichloromethane (1×20 ml), the organic layers dried over magnesium sulphate and concentrated in vacuo. The residue was crystallized from petroleum ether to afford 0.32 g (44%) of the title compound as a white solid. m.p. 132°-133° C.

### 10. 4-(Indan-1-yloxy)-1,2-dimethyl-1H-imidazo[4,5-c]pyridine

To a solution of 0.5 g (2.7 mmol) 4-chloro-1,2-dimethyl-1H-imidazo[4,5-c]pyridine and 72.6 mg (0.2 mmol) 18-crown-6 in 8 ml dimethyl sulfoxide was added dropwise a solution of 0.6 g (4.1 mmol) indan-1-ol and 0.5 g (4.1 mmol) potassium tert-butoxide in 4 ml dimethyl sulfoxide. The mixture was heated to 70° C. and stirred for 1 h. The reaction mixture was cooled to room temperature, poured into a mixture of water (50 ml)-dichloromethane (50 ml) and neutralized with potassium dihydrogen phosphate. The layers were separated, the water layer was extracted with dichloromethane (1×20 ml), the organic layers dried over magnesium sulphate and concentrated in vacuo. The residue was crystallized from cyclohexane to afford 0.12 g (16%) of the title compound as a white solid. m.p. 164°-165° C.

### 11. (6-Bromo-1,2-dimethyl-1H-imidazo[4,5-c]pyridin-4-yl)-(2-ethyl-6-methyl-benzyl)-amine

A solution of 5.7 g (16.3 mmol) 6-Bromo-N²-(2-ethyl-6-methyl-benzyl)-N⁴-methyl-pyridine-2,3,4-triamine and concentrated chloridric acid (1.5 ml) in ethanol (100 ml) was heated to reflux. A solution of 16.6 ml (130.4 mmol) trimethyl orthoacetate in 20 ml ethanol was added dropwise and the reaction mixture was refluxed for 1 h. The solution was cooled to room temperature and poured into 300 ml water. The pH was adjusted to pH=9 by a satured sodium bicarbonate solution and extracted with ethyl acetate (3×150 ml). The organic layers were dried over magnesium sulphate and concentrated in vacuo. The residue yielded 5.5 g (90%) of the title compound as a brown oil.

### 12. 4-(2-Ethyl-6-methyl-benzylamino)-1,2-dimethyl-1H-imidazo[4,5-c]pyridine-6-carboxylic Acid Ethyl Ester

To a solution of 5.4 g (14.7 mmol) (6-Bromo-1,2-dimethyl-1H-imidazo[4,5-c]pyridin-4-yl)-(2-ethyl-6-methyl-benzyl)-amine in 500 ml ethanol were added 1.5 g (5.9 mmol) triphenylphosphine, 12.2 ml (95.4 mmol) triethylamine and 0.5 g (5.9 mmol) palladium(II) acetate. The mixture was transferred to an autoclave and carbonylated (6 bar carbon monoxide pressure, 100° C.) for 20 h. The catalyst was filtered off and the filtrate concentrated in vacuo. The residue was purifed by chromatography on silica gel (ethyl acetate/petroleum ether=2:1) to afford 1.9 g (36%) of the title compound as a grey solid. m.p.>111° C. (decomposition).

### 13. 4-(2-Ethyl-6-methyl-benzylamino)-1,2-dimethyl-1H-imidazo[4,5-c]pyridine-6-carboxylic Acid

To a solution of 1.9 g (5.2 mmol) 4-(2-ethyl-6-methyl-benzylamino)-1,2-dimethyl-1H-imidazo[4,5-c]pyridine-6-carboxylic acid ethyl ester in a mixture of 4:1 dioxane/water (125 ml) were added 750 mg (31.4 mmol) lithium hydroxide and the mixture was refluxed for 4 h. After cooling to room temperature, the reaction mixture was poured into 200 ml water and the pH adjusted to pH=4 by addition of 2M HCl. The precipitate was collected by filtration and the filtrate extracted with dichloromethane (3×50 ml). The organic layers were dried over magnesium sulphate and concentrated in vacuo. Both the residue and the precipitate afforded 1.2 g (69%) of the title compound as a beige solid. m. p. 233°-236° C.

### 14. (2-Ethyl-6-methyl-benzyl)-[6-(2-methoxy-ethoxy)-1,2-dimethyl-1H-imidazo[4,5-c]pyridin-4-yl]-amine

To a solution of 0.8 g (2.4 mmol) N²-(2-ethyl-6-methyl-benzyl)-6-(2-methoxy-ethoxy)-N⁴-methyl-pyridine-2,3,4-triamine in ethanol (140 ml) were added 80 μl conc. chloridric acid and 1.8 ml (14.2 mmol) trimethyl orthoacetate. The reaction mixture was refluxed overnight, the volume reduced to the half and the solution neutralized with saturated aqueous sodium bicarbonate solution. After extraction with ethyl acetate (4×50 ml), the organic layers were dried over magnesium sulphate and concentrated in vacuo. The residue was purified by column chromatography on silica gel (petroleum ether/ethyl acetate=1:1) and crystallized from diisopropyl ether to afford 0.34 g (35%) of the title compound as a white solid. m.p. 105° C.

### 15. (2-Ethyl-6-methyl-benzyl)-(6-methoxy-1,2-dimethyl-1H-imidazo[4,5-c]pyridin-4-yl)-amine

To a solution of 1.0 g (3.3 mmol) N²-(2-ethyl-6-methyl-benzyl)-6-methoxy-N⁴-methyl-pyridine-2,3,4-triamine in ethanol (150 ml) were added 90 μl conc. chloridric acid and 2.5 ml (19.8 mmol) trimethyl orthoacetate. The reaction mixture was refluxed overnight, the volume reduced to the half and the solution neutralized with saturated aqueous sodium bicarbonate solution. After extraction with ethyl acetate (4×50 ml), the organic layers were dried over magnesium sulphate and concentrated in vacuo. The residue was purified by column chromatography on silica gel (petroleum ether/ethyl acetate=2:1) and crystallized from diisopropyl ether to afford 0.13 g (12%) of the title compound as a white solid. m.p. 106°-107° C.

### 16. 4-(2-Ethyl-6-methyl-benzylamino)-2-methyl-1H-imidazo[4,5-c]pyridine-6-carboxylic Acid Dimethyl-amide

A solution of 1.8 g (3.8 mmol) 1-benzyloxymethyl-4-(2-ethyl-6-methyl-benzylamino)-2-methyl-1H-imidazo[4,5-c]pyridine-6-carboxylic acid dimethylamide and 2.4 g (38 mmol) ammonium formiate in 30 ml ethanol was hydrogenated over 0.3 g 10% Pd/C (1 bar H₂) at 70° C. for 1 h. The reaction mixture was filtered on celite and the filtrate concentrated in vacuo. The residue was dissolved in dichloromethane, poured into water and the layers were separated. The aqueous layer was extracted with dichloromethane (3×50 ml), the organic layers dried over magnesium sulphate and concentrated in vacuo. The residue was crystallized from ethyl acetate/petroleum ether to afford 0.95 mg (73%) of the title compound as a white solid. m.p. 207°-208° C.

### 17. 6-Bromo-2-methyl-1H-imidazo[4,5c]pyridin-4-yl)-(2-ethyl-6-methyl-benzyl)-amine

A suspension of 7.2 g (19.7 mmol) 6-bromo-N(2)-(2-ethyl-6-methyl-benzyl)-3-nitro-pyridine-2,4-diamine in 520 ml ethanol was hydrogenated over Raney-Nickel (1 bar H₂) at room temperature for 5 h. The catalyst was filtered off and the filtrate treated with 4 g (41.2 mmol) acethyl acetone and 8.5 ml 5M HCl. The mixture was heated to 100° C. and stirred for 45 min. After cooling to room temperature, the volume was reduced to 100 ml and the remaining solution poured into water. The pH was adjusted to pH=8 by a satured sodium bicarbonate solution and extracted with dichloromethane (3×150 ml). The organic layers were dried over magnesium sulphate and concentrated in vacuo. The residue afforded 6.4 g (90%) of the title compound as a brown foam.

Starting Compounds and Intermediates

### A. 2,6-Dibromopyridin N-oxide

A solution of 25 g (0.1 mol) 2,6-dibromopyridine in trifluoroacetic acid (125 ml, 1.6 mol) was heated to 40° C. and 26.6 ml hydrogen peroxide were slowly added. After the addition was completed, the temperature was heated to 95° C. and the mixture stirred for 4 h. The reaction mixture was cooled to room temperature and poured into 600 ml water. The precipitate was collected by filtration and dried in vacuo to afford 20.7 g (78%) of the title compound as a beige solid. m. p. 190°-192° C.

### B. 2,6-Dibromo-4-nitro-pyridin N-oxide

To a mixture of concentrated nitric acid (25 ml) and concentrated sulphuric acid (57 ml) were cautiously added 20.7 g (81.7 mmol) 2,6-dibromopyridin N-oxide (exothermic reaction !) and the reaction mixture was heated to 60° C. After stirring for 20 h, the reaction mixture was cooled to room temperature and cautiously poured into 520 g of crushed ice. The precipitate was filtered off and dried in vacuo to afford 22.7 g (93%) of the title compound as a yellow solid. m. p. 206°-207° C.

### C. 2,6-Dibromo-4-nitro-pyridine

To a suspension of 22.7 g (76.2 mmol) 2,6-dibromo-4-nitro-pyridin N-oxide in chloroform (120 ml) was added dropwise phosphorus trichloride (20 ml, 229 mmol) at 5° C. The reaction mixture was heated to 75° C. and refluxed for 4 d. After the reaction was completed, the mixture was cautiously poured into 600 ml ice-water and the solution neutralized with sodium bicarbonate. The layers were separated and the water-layer was extracted with chloroform (3×150 ml). The organic layers were dried over magnesium sulphate and concentrated in vacuo. The residue was purified by chromatography on silica gel (petroleum ether/ethyl acetate=4:1) to give 15.5 g (72%) of the title compound as a white solid. m.p. 122°-123° C.

### D. (2,6-Dibromo-4-pyridin-4-yl)-methylamine

A solution of 15.4 g (54.8 mmol) 2,6-dibromo-4-nitro-pyridine and 25 ml methylamine (2M in THF) was transferred to an autoclave and heated at 80° C. for 90 min. After cooling to room temperature, the reaction mixture was poured into 200 ml water. The precipitate was collected by filtration and crystallized from diisopropyl ether to afford 9.8 g (67%) of the title compound as a light-yellow solid. m. p. 203°-206° C.

### E. 2,6-Dibromo-4-nitramino-pyridine

9.8 g (35.1 mmol) (2,6-Dibromo-4-pyridin-4-yl)-methylamine were carefully added to concentrated sulphuric acid (20 ml). The mixture was cooled to 0° C. and 7.6 ml concentrated nitric acid were added dropwise while the inside temperature was maintained below 10° C. with an acetone, dry ice bath. After stirring for 30 min., the reaction mixture was poured into 300 ml ice-water and extracted with dichloromethane (3×100 ml). The organic layers were dried over magnesium sulphate and concentrated in vacuo. The residue yielded 10.9 g (95%) of the title compound as a yellow oil.

### F. (2,6-Dibromo-3-nitro-4-pyridin-4-yl)-methylamine

10.0 g 2,6-Dibromo-4-nitramino-pyridine were carefully added to 110 ml concentrated sulphuric acid and the solution was heated at 100° C. for 30 min. After cooling to room temperature, the reaction mixture was poured into 400 ml ice-water and neutralized with 10 M sodium hydroxide (˜400 ml). The precipitate was collected by filtration and dried in vacuo at T=40° C. to afford 8.8 g (81%) of the title compound as a yellow solid. m.p. 121°-127° C.

### G. 6-Bromo-N-2-(2-ethyl-6-methyl-benzyl)-N-4-methyl-3-nitro-pyridine-2,4-diamine

To a solution of 5.9 g (39.6 mmol) 2-ethyl-6-methyl-benzylamine in acetonitrile (200 ml) were added 6.9 ml (53.8 mmol) triethylamine and the mixture was heated at 75° C. for 45 min. A solution of 8.8 g (28.3 mmol) 2,6-dibromo-3-nitro-4-pyridin-4-yl-methyl-amine in 100 ml acetonitrile was added dropwise and the reaction mixture was stirred at 75° C. for 6 h. The reaction was cooled to room temperature, the precipitate collected by filtration and crystallized from ethanol (200 ml) to afford 6.8 g (63%) of the title compound as a yellow solid. m.p. 148°-150° C.

### H. 6-Bromo-N-2-(2-ethyl-6-methyl-benzyl)-N-4-methyl-pyridine-2,3,4-triamine

A suspension of 6.3 g (16.7 mmol) 6-Bromo-N²-(2-ethyl-6-methyl-benzyl)-N⁴-methyl-3-nitro-pyridine-2,4-diamine in 1 L ethanol was hydrogenated over Raney-Nickel (1 bar H₂) for 30 min. The catalyst was filtered off and the filtrate concentrated in vacuo. The residue yielded 5.9 g (100%) of the title compound as a brown oil.

### I. Methyl-(3-nitro-pyridin-4-yl)-amine

To a suspension of 21.9 g (0.14 mol) 4-chloro-3-nitro-pyridine in 100 ml dichloromethane were cautiously added 62 ml (0.71 mol) methyl amine (40%-solution in water) and the reaction was heated to 40° C. After stirring for 1 h, the reaction mixture was poured into water (100 ml), the precipitate collected by filtration and dried in vacuo to afford 20 g (91%) of the title compound as a yellow solid. m.p. 159°-162° C.

### J. 2-Chloro-N-4-methyl-pyridine-3,4-diamine

0.61 g (4.0 mmol) Methyl-(3-nitro-pyridin-4-yl)-amine were dissolved in 3.5 ml conc. chloric acid and the mixture heated to 90° C. A suspension of 3.5 g (15 mmol) stannous chloride in conc. chloric acid (4 ml) was slowly added and the reaction was refluxed for 3 h. The suspension was cooled to room temperature, poured into water (75 ml) and extracted with dichloromethane (2×30 ml). The organic layers were dried over magnesium sulphate and concentrated in vacuo to afford 0.60 g (95%) of the title compound as a white solid. M. p. 168°-169° C.

### K. 4-Chloro-1,2-dimethyl-1H-imidazo[4,5-c]pyridine

5.0 g (31.7 mmol) 2-Chloro-N⁴-methyl-pyridine-3,4-diamine were suspended in 25 ml acetic anhydride and the suspension was heated at 50° C. for 1 h. The reaction was poured into water (50 ml) and concentrated in vacuo. The residue was suspended in 50 ml water and the precipitate collected by filtration to give 4.6 g (80%) of the title compound as a beige solid. m. p. 197°-198° C.

### L. N²-(2-Ethyl-6-methyl-benzyl)-6-(2-methoxy-ethoxy)-N⁴-methyl-3-nitro-pyridine-2,4-diamine

0.38 g (9.3 mmol) Sodium hydride were cautiously added to 14 ml (177 mmol) 2-methoxy-ethanol (exothermic reaction !) and the reaction was stirred at 0° C. for 45 min. 1.0 g (2.6 mmol) 6-Bromo-N²-(2-ethyl-6-methyl-benzyl)-N⁴-methyl-3-nitro-pyridine-2,4-diamine were added in portions and the reaction mixture was refluxed for 90 min. The reaction was cooled to room temperature and poured into water. The precipitate was filtered off and dried in vacuo to afford 976 mg (100%) of the title compound as a yellow solid. m.p. 135°-136° C.

### M. N²-(2-Ethyl-6-methyl-benzyl)-6-methoxy-N⁴-methyl-3-nitro-pyridine-2,4-diamine

To a suspension of 1.5 g (3.9 mmol) 6-bromo-N²-(2-ethyl-6-methyl-benzyl)-N⁴-methyl-3-nitro-pyridine-2,4-diamine in 30 ml methanol were added 2.7 ml (14 mmol) sodium methanolate (30% in methanol) and the reaction was refluxed overnight. The suspension was cooled to room temperature and poured into water (60 ml). The precipitate was filtered off and dried in vacuo to afford 1.1 g (90%) of the title compound as a yellow solid. m.p. 166°-167° C.

### N. N²-(2-Ethyl-6-methyl-benzyl)-6-(2-methoxy-ethoxy)-N⁴-methyl-pyridine-2,3,4-triamine

A suspension of 0.9 g (2.4 mmol) N²-(2-ethyl-6-methyl-benzyl)-6-(2-methoxy-ethoxy)-N⁴-methyl-3-nitro-pyridine-2,4-diamine in 140 ml ethanol was hydrogenated over Raney-Nickel for 5 h. The catalyst was filtered off and the filtrate was concentrated in vacuo. The residue afforded 0.8 g (100%) of the title compound as a green oil.

### O. N²-(2-Ethyl-6-methyl-benzyl)-6-methoxy-N⁴-methyl-pyridine-2,3,4-triamine

A suspension of 1.1 g (3.3 mmol) N²-(2-ethyl-6-methyl-benzyl)-6-methoxy-N⁴-methyl-3-nitro-pyridine-2,4-diamine in 150 ml ethanol was hydrogenated over Raney-Nickel for 7 h. The catalyst was filtered off and the filtrate was concentrated in vacuo. The residue afforded 1.0 g (100%) of the title compound as a green oil.

### P. 2,6-Dibromo-pyridin-4-ylamine

A solution of 15.3 g (54.3 mmol) 2,6-dibromo-4-nitro-pyridine and 18.5 ml (271.5 mmol) 25% aq. ammonia in 40 ml tetrahydrofurane was transferred to an autoclave and heated at 95° C. for 2.5 h. After cooling to room temperature, the reaction was poured into water (200 ml) and extracted with dichloromethane (3×200 ml). The organic layers were dried over magnesium sulphate and concentrated in vacuo. The residue was crystallized from ethyl acetate/petroleum ether to afford 9.6 g (70%) of the title compound as a yellow solid. m.p. 184°-186° C.

### Q. 2,6-Dibromo-3-nitro-pyridin-4-ylamine

A solution of 9.5 g (37.7 mmol) 2,6-dibromo-pyridin-4-ylamine in 15.3 ml concentrated sulphuric acid was cooled to 0° C. and conc. nitric acid (6.1 ml) was cautiously added. The mixture was cooled to room temperature and stirred for 45 min. The reaction was poured into ice-water (150 ml), the precipitate filtered off and washed with ethyl acetate. 8.2 g (27.6 mmol) of the yellow solid were carefully added to 82 ml conc. sulphuric acid and the reaction heated at 100° C. for 45 min. The reaction mixture was cooled to room temperature and poured into ice. The precipitate was collected by filtration and dried in vacuo at 40° C. to afford 7.3 g (91%) of the title compound as a yellow solid. m.p. 140° C.

### R. 6-Bromo-N(2)-(2-ethyl-6-methyl-benzyl)-3-nitro-pyridine-2,4-diamine

To a solution of 6.9 g (23.4 mmol) 2,6-dibromo-3-nitro-pyridin-4-ylamine in 120 ml acetonitrile were added 5.7 ml triethylamine and the mixture was heated to 75° C. A solution of 4.9 g (32.7 mmol) 2-ethyl-6-methyl-benzylamine in acetonitrile (100 ml) was added dropwise and the reaction stirred for 1 h. After cooling to room temperature, the solvent was removed and the residue crystallized from diethyl ether to afford 7.3 g (86%) of the title compound as a yellow solid. m.p. 126°-128° C.

### S. (1-Benzyloxymethyl-6-bromo-2-methyl-1H-imidazo[4,5-c]pyridin-4-yl)-(2-ethyl-6-methyl-benzyl)-Amine

5.7 g (15.8 mmol) (6-Bromo-2-methyl-1H-imidazo[4,5-c]pyridin-4-yl)-(2-ethyl-6-methyl-benzyl)-amine were dissolved in 23 ml dimethylformamide and the solution was cooled to 0° C. 420 mg (17.4 mmol) sodium hydride were added in portions, the mixture was stirred for 30 min. and then 3.5 ml (23.7 mmol) benzyl chloromethyl ether were cautiously added. After 90 min., the reaction was poured into a satured sodium bicarbonate solution (30 ml) and extracted with dichloromethane (3×20 ml). The organic layers were dried over magnesium sulphate and concentrated in vacuo. The residue was purified by chromatography on silica gel (petroleum ether/ethyl acetate=5:4) to afford 3.6 g (47%) of the title compound as a brown oil.

### T. 1-Benzyloxymethyl-4-(2-ethyl-6-methyl-benzylamino)-2-methyl-1H-imidazo[4,5c]pyridine-6-carboxylic Acid Ethyl Ester

To a solution of 3.5 g (7.3 mmol) (1-benzyloxymethyl-6-bromo-2-methyl-1H-imidazo[4,5-c]pyridin-4-yl)-(2-ethyl-6-methyl-benzyl)-amine in 65 ml ethanol were added 12.5 ml triethylamine and 0.5 g (0.7 mmol) bis-triphenylphosphine-Pd(II)-chloride. The mixture was transferred to an autoclave and carbonylated (6 bar carbon monoxide pressure, 100° C.) for 18 h. The catalyst was filtered off and the filtrate concentrated in vacuo. The residue was purified by chromatography on silica gel (petroleum ether/ethyl acetate=1:1) to afford 2.7 g (79%) of the title compound as a yellow oil.

### U. 1-Benzyloxymethyl-4-(2-ethyl-6-methyl-benzylamino)-2-methyl-1H-imidazo[4,5c]pyridine-6-carboxylic Acid

To a solution of 2.6 g (5.5 mmol) 1-benzyloxymethyl-4-(2-ethyl-6-methyl-benzylamino)-2-methyl-1H-imidazo [4,5-c]pyridine-6-carboxylic acid ethyl ester in a mixture of 140 ml dioxane-water (4:1) were added 820 mg lithium hydroxide and the mixture was refluxed for 1 h. After cooling to room temperature, the dioxane was evaporated in vacuo and the aqueous solution adjusted to pH=8 by addition of 2.5 M HCl. The precipitate was filtered and dried in vacuo at 40° C. to afford 2.2 g (92%) of the title compound as a white solid. m.p. 196° C.

### V. 1-Benzyloxymethyl-4-(2-ethyl-6-methyl-benzylamino)-2-methyl-1H-imidazo[4,5-c]pyridine-6-carboxylic Acid Dimethylamide

To a suspension of 2.1 g (4.8 mmol) 1-benzyloxymethyl-4-(2-ethyl-6-methyl-benzylamino)-2-methyl-1H-imidazo[4,5-c]pyridine-6-carboxylic acid in 70 ml dichloromethane were added 2.3 g (7.2 mmol) O-(1H-benzotriazol-1-yl)-N,N,N′,N′-tetramethyluronium tetrafluoroborate (TBTU) and the mixture was stirred at room temperature overnight. 9.6 ml (19.2 mmol) Dimethylamine (2M in THF) were added and the reaction was stirred for 6 h. The mixture was poured into water and extracted with dichloromethane. The organic layers were dried over magnesium sulphate and concentrated in vacuo. The residue was purified by chromatography on silica gel (dichloromethane/methanol=20:1) to afford 2.4 g (85%) of the title compound as a yellow oil.

Commercial Utility

The compounds of the formula 1 and their salts have valuable pharmacological properties which make them commercially utilizable. In particular, they exhibit marked inhibition of gastric acid secretion and an excellent gastric and intestinal protective action in warm-blooded animals, in particular humans. In this connection, the compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the compounds according to the invention surprisingly prove to be clearly superior to the compounds known from the prior art in various models in which the antiulcerogenic and the antisecretory properties are determined. On account of these properties, the compounds of the formula 1 and their pharmacologically acceptable salts are outstandingly suitable for use in human and veterinary medicine, where they are used, in particular, for the treatment and/or prophylaxis of disorders of the stomach and/or intestine.

A further subject of the invention are therefore the compounds according to aspect 3 the invention for use in the treatment and/or prophylaxis of the abovementioned diseases.

The invention likewise includes the use of the compounds according to aspect 3 of the invention for the production of medicaments which are employed for the treatment and/or prophylaxis of the above mentioned diseases.

The invention furthermore includes the use of the compounds according to aspect 3 of the invention for the treatment and/or prophylaxis of the abovementioned diseases.

A further subject of the invention are medicaments which comprise one or more compounds of the formula 1 according to aspect 3 of the invention and/or their pharmacologically acceptable salts.

The medicaments are prepared by processes which are known per se and familiar to the person skilled in the art. As medicaments, the pharmacologically active compounds according to the invention (=active compounds) are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously.

In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the compounds according to the invention and/or their salts are to be used for the treatment of the abovementioned diseases, the pharmaceutical preparations can also contain one or more pharmacologically active constituents of other groups of medicaments, for example: tranquillizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, mezlocillin, ampicillin, amoxicillin, cefalothin, cefoxitin, cefotaxime, imipenem, gentamycin, amikadn, erythromycin, ciprofloxacin, metronidazole, clarithromycin, azithromycin and combinations thereof (for example clarithromycin+metronidazole).

In view of their excellent gastric and intestinal protection action, the compounds of formula 1 are suited for a free or fixed combination with those medicaments (e.g. certain antiinflammatories and antirheumatics, such as NSAIDs), which are known to have a certain ulcerogenic potency. In addition, the compounds of formula 1 are suited for a free or fixed combination with motility-modifying drugs.

Pharmacology

The excellent gastric protective action and the gastric acid secretion-inhibiting action of the compounds according to aspect 3 of the invention can be demonstrated in investigations on animal experimental models. The compounds according to the invention investigated in the model mentioned below have been provided with numbers which correspond to the numbers of these compounds in the examples.

Testing of the Secretion on the Perfused Rat Stomach

In Table A which follows, the influence of the compounds of the formula 1 according to aspect 3 of the invention on the pentagastrin-stimulated acid secretion of the perfused rat stomach after intraduodenal

TABLE A

| | Dose | Inhibition of |
|---|---|---|
| | (μmol/kg) | acid secretion |
| No. | i.d. | (%) |
| 2 | 1.0 | >30 |
| 3 | 1.0 | >30 |
| 4 | 1.0 | >30 |
| 16 | 1.0 | >30 |

Methodology

The abdomen of anesthetized rats(CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01 N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion.

The body temperature of the animals was kept at a constant 37.8-38° C. by infrared irradiation and heat pads (automatic, stepless control by means of a rectal temperature sensor).

1. A method of preventing or treating a gastrointestinal disorder in a patient comprising administering to a patient in need thereof a therapeutically effective amount of a compound of the formula 1, 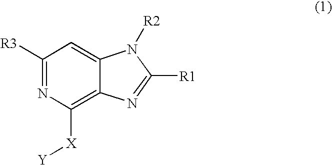
in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, or fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4Calkoxy-1-4C-alkoxy or the group —CO—NR31R32,
where
R31 is hydrogen, hydroxyl, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
wherein
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
or Y denotes the group gp 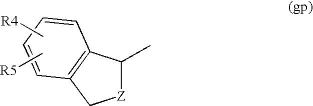
wherein
Z has the meaning —CHR8- or —CHR8-CHR9-
where in Ar and/or in the group gp
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
and wherein
aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano,
or a pharmacologically acceptable salt thereof.

2. (canceled)

3. A compound of the formula 1, 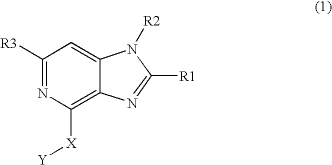
in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, or fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4Calkoxy-1-4C-alkoxy or the group —CO—NR31R32,
where
R31 is hydrogen, hydroxyl, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
wherein
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
or Y denotes the group gp 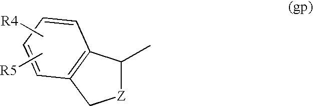
wherein
Z has the meaning —CHR8- or —CHR8-CHR9-
where in Ar and/or in the group gp
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
and wherein
aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano,
with the proviso that R1 does not have the meaning hydrogen when
R2 is hydrogen, 1-4C-alkyl, 1-4C-alkoxy-1-4C-alkyl or cyclopropylmethyl,
R3 is hydrogen or halogen,
X is O (oxygen) or NH,
Y denotes CH₂—Ar
wherein
Ar is phenyl, naphthyl or benzothienyl substituted by R4 and R5
wherein
R4 is hydrogen, halogen, trifluoromethyl or nitro,
R5 is hydrogen, halogen or trifluoromethyl,
or a salt thereof.

4. The compound of the formula 1 as claimed in 3,
in which
R1 is 1-4C-alkyl,
R2 is hydrogen or 1-4C-alkyl,
R3 is hydrogen, halogen, carboxyl, 1-4C-alkoxycarbonyl, 1-4C-alkoxy, 1-4C-alkoxy-1-4C-alkoxy or the group —CO—NR31R32,
where
R31 is hydrogen, 1-4C-alkyl or hydroxy-1-4C-alkyl and
R32 is hydrogen or 1-4C-alkyl,
X is O (oxygen) or NH and
Y has either the meaning —CH₂—Ar
wherein
Ar is a phenyl substituted by R4 and R5,
or Y denotes the group gp 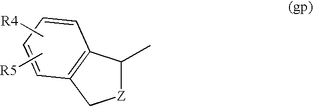
wherein
Z has the meaning —CHR8-,
where in Ar and/or in the group gp
R4 is hydrogen, 1-4C-alkyl or halogen,
R5 is hydrogen or 1-4C-alkyl,
R8 is hydrogen
or a salt thereof.

5. The compound as claimed in claim 3, characterized by the formula 1a 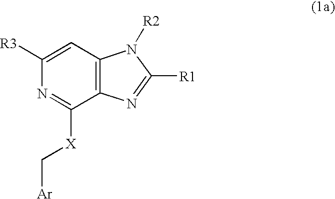
in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, or fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4C-alkoxy-1-4C-alkoxy or the group —CO—NR31R32,
where
R31 is hydrogen, hydroxyl, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
where
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen, aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano,
with the proviso that R1 does not have the meaning hydrogen when
R2 is hydrogen, 1-4C-alkyl, 1-4C-alkoxy-1-4C-alkyl or cyclopropylmethyl,
R3 denotes hydrogen or halogen,
X is O (oxygen) or NH,
Ar is phenyl, naphthyl or benzothienyl substituted by R4 and R5
wherein
R4 is hydrogen, halogen, trifluoromethyl or nitro,
R5 is hydrogen, halogen or trifluoromethyl,
or a salt thereof.

6. The compound as claimed in claim 3, characterized by the formula 1b 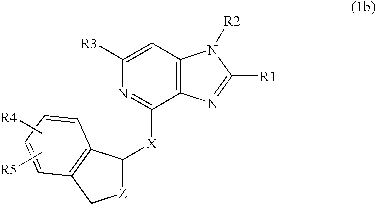
in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, or fluoro-2-4C-alkyl,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy, 1-4C-alkoxy-1-4C-alkoxy or the group —CO—NR31R32,
where
R31 is hydrogen, hydroxyl, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
X is O (oxygen) or NH and
Z has the meaning —CHR8- or —CHR8-CHR9-
where
R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
and wherein
aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano,
or a salt thereof.

7. The compound as claimed in claim 5, characterized by the formula 1a-1, 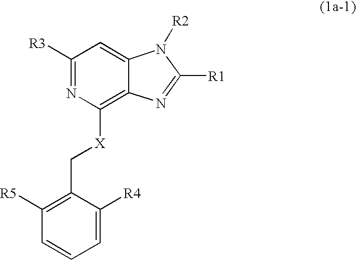
in which
R1 is hydrogen or 1-4C-alkyl,
R2 is hydrogen or 1-4C-alkyl,
R3 is hydrogen, halogen, carboxyl, 1-4C-alkoxycarbonyl, hydroxy-1-4C-alkyl, 1-4C-alkoxy-1-4C-alkyl, 1-4C-alkoxy, 1-4C-alkoxy-1-4C-alkoxy or the group —CO—NR31R32,
where
R31 is hydrogen, 1-7C-alkyl, hydroxy-1-4C-alkyl or 1-4C-alkoxy-1-4C-alkyl and
R32 is hydrogen or 1-7C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl, 1-4C-alkoxy or halogen and
X is O (oxygen) or NH,
with the proviso that R1 does not have the meaning hydrogen when
R2 is hydrogen or 1-4C-alkyl,
R3 is hydrogen or halogen,
X is O (oxygen) or NH,
R4 is hydrogen, halogen or trifluoromethyl,
R5 is hydrogen or halogen,
or a salt thereof.

8. The compound of the formula 1a-1 as claimed in claim 7,
in which
R1 is 1-4C-alkyl,
R2 is hydrogen or 1-4C-alkyl,
R3 is hydrogen, halogen, carboxyl, 1-4C-alkoxycarbonyl, 1-4C-alkoxy, 1-4C-alkoxy-1-4C-alkoxy or the group —CO—NR31R32,
where
R31 is hydrogen, 1-4C-alkyl or hydroxy-1-4C-alkyl and
R32 is hydrogen or 1-4C-alkyl,
R4 is hydrogen, 1-4C-alkyl or halogen
R5 is hydrogen, 1-4C-alkyl or halogen,
X is O (oxygen) or NH,
or a salt thereof.

9. The compound as claimed in claim 6, characterized by the formula 1b-1 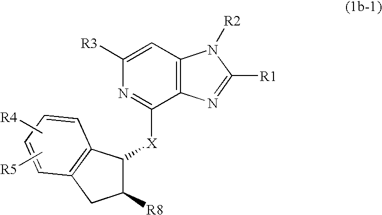
in which
R1 is hydrogen or 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy-1-4C-alkoxy-1-4C-alkyl, 1-4C-alkoxy, 1-4C-alkoxy-1-4C-alkoxy or the group —CO—NR31R32,
where
R31 is hydrogen, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen,
R8 is hydrogen, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
X is O (oxygen) or NH,
or a salt thereof.

10. The compound of the formula 1b-1 as claimed in claim 9,
in which
R1 is 1-4C-alkyl,
R2 is hydrogen or 1-4C-alkyl,
R3 is hydrogen, halogen, carboxyl, 1-4C-alkoxycarbonyl, 1-4C-alkoxy-1-4C-alkyl, 1-4C-alkoxy, 1-4C-alkoxy-1-4C-alkoxy or the group —CO—NR31R32,
where
R31 is hydrogen, 1-4C-alkyl, hydroxy-1-4C-alkyl or 1-4C-alkoxy-1-4C-alkyl and
R32 is hydrogen or 1-4C-alkyl,
R4 is hydrogen, 1-4C-alkyl, 1-4C-alkoxy or halogen,
R5 is hydrogen,
R8 is hydrogen or hydroxyl,
X is O (oxygen) or NH,
or a salt thereof.

11. The compound of the formula 1b-1 as claimed in claim 9,
in which
R1 is 1-4C-alkyl,
R2 is 1-4C-alkyl,
R3 is hydrogen,
R4 is hydrogen,
R5 is hydrogen,
R8 is hydrogen,
X is O (oxygen),
or a salt thereof.

12. A pharmaceutical composition comprising a compound as claimed in claim 3 and/or a pharmacologically acceptable salt thereof together with a pharmaceutically acceptable auxiliary and/or excipient.

13.-14. (canceled)
